(19) United States
(12) Patent Application Publication
(10) Pub. No.: US 2007/0213625 A1
(43) Pub. Date: Sep. 13, 2007

(54) Cardiac output monitoring system and method using electrical impedance plythesmography

(75) Inventors: Vishwanath Panduranga Nayak, Mysore (IN); Tejas Kumar Bengali, Mysore (IN); Raj Agarwal, Mysore (IN); Nagarajan Ravindran, Mysore (IN); Swarupanand Sewalkar, Mysore (IN)

Correspondence Address: THE LAW OFFICES OF ANDREW D. FORTNEY, PH.D., P.C., 401 W FALLBROOK AVE STE 204, FRESNO, CA, 93711-5835, US

(73) Assignee: Larson & Toubro Limited

(21) Appl. No.: 11/510,149

(22) Filed: Aug. 24, 2006

(30) Foreign Application Priority Data
Feb. 24, 2006 (IN) 304/CHE/2006

(51) Int. Cl. A61B 5/02; A61B 5/05

(52) U.S. Cl. 600/506; 600/547; 600/504

(57) ABSTRACT

The present invention provides a noninvasive and portable medical monitoring system for monitoring the change in time of the electrical impedance of a portion of a living body, such as the lungs or the brain with an inbuilt data acquisition system and a PC motherboard. The present invention also provides a computer implementable method for monitoring and measurement of cardiac output and blood flow index using impedance plythesmographic techniques.

### FIELD OF THE INVENTION

The present invention relates generally to noninvasive medical monitoring systems and, more particularly, to a method and device for monitoring the change in time of the electrical impedance of a portion of a living body, such as the lungs or the brain. More particularly, the present invention relates to a portable monitoring system for measurement of cardiac output and blood flow index using impedance plythesmographic techniques.

### BACKGROUND OF THE INVENTION

An accurate monitoring and measurement of cardiac output has long been a clinical and research goal. Several methods are known in the art for the monitoring and measurement of cardiac output including both direct and indirect methods. The measurement and monitoring of cardiac output has been known for over seventy years. A representative and not an exhaustive list are given below in respect of the various methods employed for measurement and monitoring of cardiac output.

Direct methods for measurement and monitoring of cardiac output are generally more accurate but are largely restricted to research laboratories due to the invasive or traumatic procedures, which need to be employed. Indirect methods such as the steady-state Fick oxygen uptake, the transient indicator dilution method, and anemometry are less invasive but are not very accurate.

Of the less invasive indirect methods, the transient indicator dilution procedure using iced liquids injected through the lumen of a Swan-Ganz catheter is currently the most frequently employed clinical method. This method requires the least amount of specialized equipment is portable to the patent's bedside and can be repeated often. However, the transient indicator dilution procedure requires a specially trained physician to thread an expensive catheter through the right side of the heart and into the pulmonary artery. During long term monitoring, infection at the site of catheter insertion and damage to the blood vessels of the lung are constant hazards. The Swan-Ganz catheters may also need to be repositioned or replaced after a few days of use. Accuracy and repeatability of the thermal dilution Swan-Ganz method are substantially low, even under precisely controlled laboratory conditions.

Non-invasive indirect methods also includes the ballistocardiography method which requires a patient to lie motionless on a large inertial platform, the soluble gas uptake method which requires a patient to sit in a small chamber for many minutes and the impedance plethysmography method which measures small changes in electrical impedance on the surface of the chest. The first two non-invasive methods are not readily utilized because the special equipment needed is extremely large and inconvenient to use. In impedance plethysmography, accuracy is difficult to obtain and is thus not normally preferred.

Representative heart imaging techniques include 2-D cine-angiography and 2D echo-cardiography wherein a series of x-ray or ultrasound images of the beating heart are measured to determine left ventricle systolic and diastolic volumes. 3-D ECG-gated MRI and radioactive imaging methods where many images of the heart are made during particular phases of the cardiac cycle can also be employed. These methods require large, expensive equipment, and measurements are time consuming and require the efforts of several highly trained specialists to obtain and interpret results.

A significant problem associated with heart diseases is the fluid buildup such as acute edema of the lungs. Since these fluids are electrically conductive, changes in their volume can be detected by the technique of impedance plethysmography, in which the electrical impedance of a part of the body is measured by imposing an electrical current across the body and measuring the associated voltage difference. For example, experiments with dogs (R. V. Luepker et al., American Heart Journal, Vol. 85, No. 1, pp 83-93, January 1973) have shown a clear relationship between the transthoracic electrical impedance and the change in pulmonary fluid volume.

Several methods are known in the art for monitoring of pulmonary edema using two electrodes, one either side of the biological object. However, such methods have proved to be unfit for prolonged monitoring due to the drift of skin-to-electrode contact layer resistance. This drift is due to ions from sweat and skin penetrating the electrolytic paste of the electrode, and the wetting of the epidermis, over the course of several hours. A method for overcoming this problem was developed by Kubicek et al. (Annals of the New York Academy of Sciences, 1970, 170(2):724-32; U.S. Pat. No. 3,340,867, reissued as Re. Pat. No. 30,101). Related U.S. patents include Asrican (U.S. Pat. No. 3,874,368), Smith (U.S. Pat. No. 3,971,365), Matsuo (U.S. Pat. No. 4,116,231) and Itoh (U.S. Pat. No. 4,269,195). The method of Kubicek et al. uses a tetrapolar electrode system whereby the outer electrodes establish a current field through the chest. The inner voltage pickup electrodes are placed as accurately as is clinically possible at the base of the neck and at the level of the diaphragm. This method regards the entire portion of the chest between the electrodes as a solid cylinder with uniform parallel current fields passing through it. However, because this system measures the impedance of the entire chest, and because a large part of the electrical field is concentrated in the surface tissues, this method is not sufficiently specific for measuring liquid levels in the lungs and has low sensitivity: 50 ml per Kg of body weight (Y. R. Berman, W. L. Schutz, Archives of Surgery, 1971.V.102:61-64). It should be noted that such sensitivity has proved to be insufficient for obtaining a significant difference between impedance values in patients without pulmonary edema to those with an edema of average severity (A. Fein et al., Circulation, 1979, 60(5):1156-60). In their report on the conference in 1979 concerning measuring the change in the liquid level in the lungs (Critical Care Medicine, 1980, 8(12):752-9), N. C. Staub and J. C. Hogg summarize the discussion on the reports concerning the reports on the method of Kubicek et al. for measuring thoracic bio-impedance. They conclude that the boundaries of the normal values are too wide, and the sensitivity of the method is lower than the possibilities of clinical observation and radiological analysis, even when the edema is considered to be severe. It is indicative that, in a paper six years later by N. C. Staub (Chest. 1986, 90(4):588-94), this method is not mentioned at all. Other problems with this method include the burdensome nature of the two electrodes tightly attached to the neck, and the influence of motion artifacts on the impedance readings received.

Another method for measuring liquid volume in the lungs is the focusing electrode bridge method of Severinghaus (U.S. Pat. No. 3,750,649). This method uses two electrodes located either side of the thorax, on the left and right axillary regions. Severinghaus believed that part of the electrical field was concentrated in surface tissues around the thorax and therefore designed special electrodes to focus the field through the thorax. This method does not solve the problems associated with the drift in the skin-to-electrode resistance described above. An additional problem is the cumbersome nature of the large electrodes required. It is indicative that the article by Staub and Hogg, describing the 1979 conference, mentions that the focusing bridge transthoracic electrical impedance device was not discussed, despite the presence of its developer at the conference. A review by M. Miniati et al. (Critical Care Medicine, 1987, 15(12):1146-54) characterizes both the method of Kubicek et al. and the method of Severinghaus as “insufficiently sensitive, accurate, and reproducible to be used successfully in the clinical setting” (p. 1146).

Toole et al., in U.S. Pat. No. 3,851,641, addresses the issue of electrode drift by measuring the impedance at two different frequencies. However, their method is based on a simplified equivalent circuit for the body in which the resistances and capacitances are assumed to be independent of frequency. Pacela, in U.S. Pat. No. 3,871,359, implicitly addresses the issue of electrode drift by measuring two impedances across two presumably equivalent parts of a body, for example, a right and a left arm or a right and a left leg, and monitoring the ratio between the two impedances. His method is not suitable for the monitoring of organs such as the lungs, which are not symmetric, or the brain, of which the body has only one. Other notable recent work in measuring the impedance of a portion of the body includes the tomographic methods and apparatuses of Bai et al. (U.S. Pat. No. 4,486,835) and Zadehkoochak et al. (U.S. Pat. No. 5,465,730). In the form described, however, tomographic methods are based on relatively instantaneous measurements, and therefore are not affected by electrode drift. If tomographic methods were to be used for long-term monitoring of pulmonary edema, they would be as subject to electrode drift problems as the other prior art methods.

As seen above, it is important to estimate cardiac output. Noninvasive estimates of cardiac output (CO) can be obtained using impedance cardiography. Strictly speaking, impedance cardiography, also known as thoracic bio-impedance or impedance plethysmography, is used to measure the stroke volume of the heart. Cardiac output is obtained when the stroke volume is multiplied by heart rate.

Heart rate is obtained from an electrocardiogram. The basic method of correlating thoracic, or chest cavity, impedance, Z_{T} (t), with stroke volume was developed by Kubicek, et al. at the University of Minnesota for use by NASA. See, e.g., U.S. Reissue Pat. No. 30,101 entitled “Impedance plethysmograph” issued Sep. 25, 1979, which is incorporated herein by reference in its entirety. The method generally comprises modeling the thoracic impedance Z_{T} (t) as a constant impedance, Z_{O}, and time-varying impedance, δZ (t). The time-varying impedance is measured by way of an impedance waveform derived from electrodes placed on various locations of the subject's thorax; changes in the impedance over time can then be related to the change in fluidic volume (i.e., stroke volume), and ultimately cardiac output.

In order to do the cardiac output measurement selection of ‘a’, ‘b’, ‘c’ and ‘x’ points is necessary on the time varying impedance graph. The ‘c’ point being the peak point, ‘a’ and ‘x’ points can be identified as the lowest points on the left and the tight side of point ‘c’ respectively. ‘b’ point can located in between ‘a’ and ‘c’ points at the start of the peak. But it can be tricky to identify these points manually and human error in judgement could mean error in diagnosing the exact condition of the patient. Hence it is important to develop better ways of identifying these points so that more accurate measurement of cardiac output can happen.

Also the existing apparatus for non-invasive cardiac output measurement are not easy to use and involve complex connections. They typically involve a conventional stand alone PC connected to plethysmography related gadgets. Which means, the equipment as a whole is cumbersome to use and cannot be moved around easily to take the equipment near a patient if required.

The existing apparatus are also limited in their capacity to do analysis based on a particular patient's data due to limitations in the software being employed as part of the apparatus.

Thus, there exists a need for an improved apparatus and method for measuring cardiac output. Such improved apparatus and method ideally be easy to use and operate, would allow the clinician to repeatedly and consistently identify the ‘a’, ‘b’, ‘c’ and ‘x’ points for accurate measurement of cardiac output and also allow repeated analysis on a patient's data for assisting the clinician in diagnosing the situation in the most accurate manner.

### OBJECTS OF THE INVENTION

One object of the invention is to provide an integrated and easy to use impedance plethysmograph apparatus

Another object of the invention is to provide accurate measurement of cardiac output by providing both intermittent and continuous cardiac output measurement modes, wherein under the continuous output measurement mode, the selection of points on the time varying impedance graph happens automatically and under the intermittent mode, the selection of points needs to be done manually

Another object of the present invention is to extract respiration rate waveform, which is another important parameter to be monitored that gives an indication of the stress condition of the patient

Another object of the present invention is to provide facility to re-analyze a patient's data after doing a first analysis by storing the patients data in the storage memory with a unique identifier for the patient enabling easy retrieval for re-analysis

Another object of the present invention is to provide low cost solution to the existing impedance plethysmograph apparatus by providing digital solutions to existing analog circuitry

Another object of the present invention is to provide an apparatus that can be used both for non-invasive cardiac output monitoring and vascular measurement monitoring

### SUMMARY OF THE INVENTION

Accordingly, the present invention provides a noninvasive and portable medical monitoring apparatus for monitoring the change in time of the electrical impedance of a portion of a living body, such as the lungs or the brain. The present invention also provides a method for monitoring and measurement of cardiac output and blood flow index using impedance plythesmographic techniques. The present invention uses a tetra polar electrode method with a TFT display unit to measure, in litres, the blood pumped by the heart at a given period of time with an option to trace vascular resistance. The present invention measures the change in the body surface impedance due to pulsetile blood flow by injecting carrier charges such as a low amplitude sinusoidal current with a high frequency such as 48 kHz and monitoring the voltage variations along the current path.

The present invention also provides facility store copies of patient information and waveforms with an unique identifier for easy retrieval and re-analysis. The invention also reduces the complex analog circuitry found in the conventional plethysmograph apparatus by using digital solutions for the same circuitry, especially in the circuitry for cal pulse generation and carrier sine wave generation.

### BRIEF DESCRIPTION OF THE DRAWINGS

FIG. 1 provides a schematic block diagram of the system of the invention.

FIG. 2 shows the block diagram of an exemplary analog system for cal pulse generation and carrier sine wave generation.

FIG. 3 shows a preferred embodiment of the digital implementation of the cal pulse generation and carrier sine wave generation of the present invention.

FIG. 4 shows the block diagram of an exemplary analog circuitry for generating dZ/dt differentiated waveform.

FIG. 5 shows a preferred embodiment of the digital implementation of the circuitry for generation of dZ/dt differentiated waveform.

FIG. 6 is a representative rate of change of impedance waveform.

### DETAILED DESCRIPTION OF THE INVENTION

The present invention relates to a medical monitoring system and more particularly to a method and portable device for monitoring volume of fluid associated with the heart. In other words, the invention is used to measure the volume of blood pumped by the heart per minute, namely the blood flow index. As these fluids are electrically conductive, charges in their volumes can be detected by the technique of impedance plythesmography wherein the electrical impedance of a part of the body is measured by imposing an electrical current across the body and measuring the associated voltage difference.

The system of the invention provides an apparatus for monitoring cardiac output using impedance plythesmographic techniques and tracing vascular resistance using a dedicated menu option. Thus different options are provided for working of the monitor. The method uses tetrapolar electrode systems. One pair of electrodes is utilised for sensing voltage drop along the current path that takes place due to changing blood flow with heart beat. The monitor of the invention is particularly advantageous since it is portable. In addition multiple keys are provided along with an optical encoder for data entry and menus selection. The display monitor can be a 10.4 inch TFT display panel and is provided with a back up power source.

The method of the invention comprises of:
1. Signal acquisition and signal conditioning.
2. Computation of cardiac output, blood flow index using the tetra polar electrode method;
3. Display of the results of the signal acquisition and conditioning, computation of the cardiac output and the blood flow index on a 10.4″ TFT monitor.

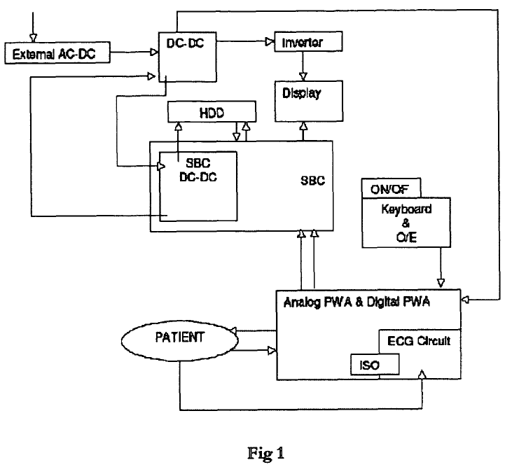

FIG. 1 shows the system block diagram. The external ac voltage (works from 95-265V 60 Hz/50 Hz) (230 V) has to be converted into a DC (12-13.8V) voltage initially as shown in the block diagram, which is fed to the DC-DC converter card and parrellely to Single Board computer DC-DC converter (SBC DC-DC). The SBC DC-DC supplies Power to the single board computer. DC-DC Converter supplies to the rest of the Boards like NICO Amp card (In block it is mentioned as analog and digital+ISO), Inverter card, Key board, Fan (to keep the temp cooling inside), because all these boards need a constant DC voltage for its operation. The SBC has a two way communication with the hard disk drive (HDD) for storing data and retrieving information from the hard disk. The DC voltage is again converted to ac voltage by the inverter for the display backlight unit as shown in the block diagram. NICO amp card is specially designed to provide 5 kV patient Isolation and less than 10 uA patient leakage current. The analog and digital PWA (Nico Amp card) are used to gather (Impedance changes) and ECG signals from the patient body, demodulate, signal condition by removing noise, amplify it and then digitize and display as a waveform on the display. The NICO Amp card also has the in built ECG generation circuit to facilitate the Nico calculation. Nico Amp card also has the on board CAL pulse generation to facilitate the user to check the calibration status of the unit without opening the it. This on board Cal Pulse gives facility to do on site calibration without any speciliased equipment to be carried for the same. There is a isolator to isolate the voltage in the ECG circuit to 5 kV. The Nico waveform is managed by a keyboard, which can keeps the waveform the following states:
start/stop
freeze/de-freeze

### The On/off key of the key board is used to start and stop the key board.

The system is designed such that power supply to display invertor is given only after sensing that SBC DC-DC has switched on, so that the display comes once the SBC has booted to give good impression of the product. Also during switch off if the on/off key is pressed for three seconds and then software senses and shut down the software and then both the DC-DC converters will shut off. The waveform from the PWA is fed back to the single board computer which then displays it on the display unit.

Signal acquisition is carried out using an acquisition board. The computation is carried out using a digital board which uses a Intel 80c251 processor and a mother board. The unit has the facility to load NICO software without opening the unit though USB port. The unit also has the Key board and mouse interface facility to type the letters in the menu and selection of the points on the waveform more accurately. The unit also has the VGA out put to connect to the external monitor as well as Project to connect bigger displays.

### Circuitry for Cal Pulse Generation and Carrier Sine Wave Generation

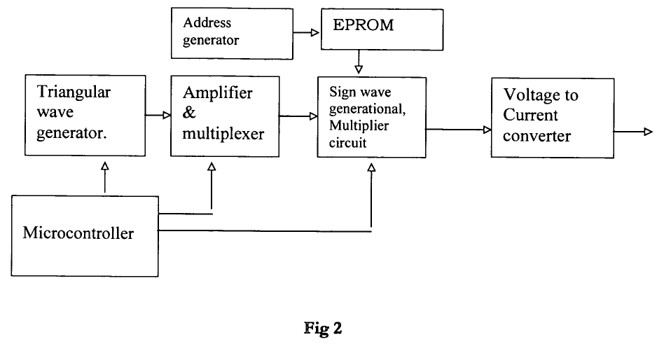

FIG. 2 shows the block diagram of an exemplary analog system for cal pulse generation and carrier sine wave generation. The sine wave current source (1) is typically EPROM driven and contains sine wave values, and generates a 48 kHz sine wave. The sine wave current generator passes the sine wave of constant amplitude through the body segment in “patient mode” with the help of an isolation X'mer and relay. The sine wave current generator also passes a modulated sine wave current (1% amp. modulation with triangular wave of 1 Hz frequency) to the calibration n/w of fixed resistor values in the calibration mode. The voltage signal developed in the ‘current’ path is sensed with the help of sensing electrodes and amplified using a differential amplifier. The high ‘Q’ band pass filter removes the super imposed noise and the output of the filter is rectified by precision rectifier and filtered to obtain a filtered (output) signal ‘Z’, that is proportional to the instantaneous electrical impedance of the body segment, under investigation.

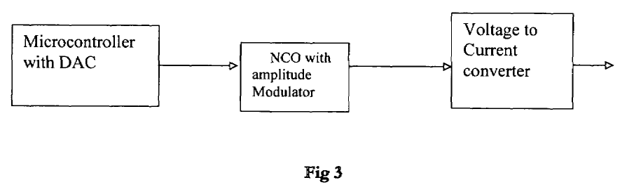

FIG. 3 shows a preferred embodiment of the digital implementation of the cal pulse generation and carrier sine wave generation of the present invention. A single micro controller with DAC will replace the triangular wave generator, amplifier & multiplexer and the microcontroller blocks shown in FIG. 3. Also the address generator and EPROM is replaced by using Numerically controlled Oscillator (NCO). Thus circuit is made much simpler and cost effective along with all the benefits of accuracy associated with digital circuits.

### Circuitry for Generating dZ/dt Differentiated Waveform

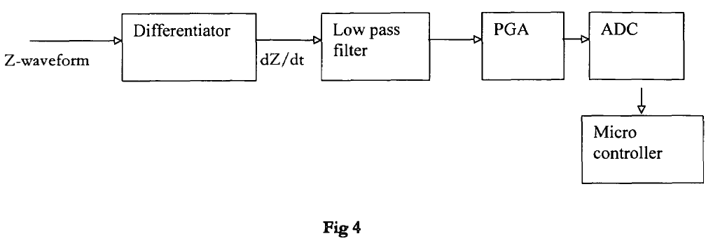

FIG. 4 shows the block diagram of an exemplary analog circuitry for generating dZ/dt differentiated waveform. This signal is attenuated and fed to ADC input of the digital circuit comprising microcontroller Intel 80-c251. The initial value of impedance (Z₀) is outputted by the controller to a 12 bit DAC, the output of which is fed to one of the inputs of differential amplifier with ‘Z’ as the other input. The differential amplifier outputs □Z(t) signal, which gives change in impedance of the body segment as a function of time. It is low pass filtered, provided programmable gain and limited to 5V amplitude and given to ADC input of digital circuit. ‘Z’ is also used to obtain dZ/dt signal with the help of a differentiator circuit (6). It is low pass filtered, provided programmable gain, limited to 5 V & given to ADC input of digital card. The CAL/PAT relay and selection of current value in the sine wave current source is controlled through the micro-controller.

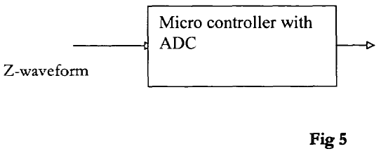

FIG. 5 shows a preferred embodiment of the digital implementation of the circuitry for generation of dZ/dt differentiated waveform. As seen in FIG. 4, the Z-waveform (which is impedance waveform from the body) is differentiated using Analog Differentiator and dZ/dt waveform is got, which is passed through LPF and Programmable gain amplifier and then analog to digital converter in an exemplary analog setup. This digital data is then given to micro controller and which will use for process the data to show as waveform on the screen and there by calculate CO. In the digital circuitry, the Z-waveform is fed directly to ADC of micro controller and converted to digital Z data waveform, which will be further processed using software techniques to generate dZ/dt. Which is further processed. Here again, the circuitry is made much simpler and Reliable.

The ECG is sensed from RA and LL of the patient with the help of surface electrodes in order to provide synchronous pulse for ensemble averaging of the IPG (impedance plethysmograph signal) signal. The signal is amplified with the help of an isolation amplifier. ‘R’ wave of ECG or on set of ‘CAL’ signal is detected with help of an adaptable threshold ‘R’ wave detector and TTL pulse synchronous with ‘R’ wave of ECG are obtained & connected to one of the input port of Micro controller. The Analog ECG is also separately connected to one of the ADC Channels. The Digital ECG will be used for displaying on the screen as well to calibrate the ECG and also to check whether ECG Quality is good. The digital card is connected to PC through serial communication link (RS232).

The selection of differential hardware parameter such as current amplifier (4 mA/2 mA/CAL), output waveforms (dZt, dZ/dt, N dZ/dt) and gain of the system (½, 1 & 2) is performed with the help of user friendly menu driven program running on the SBC.

### Cardiac Output Measurement

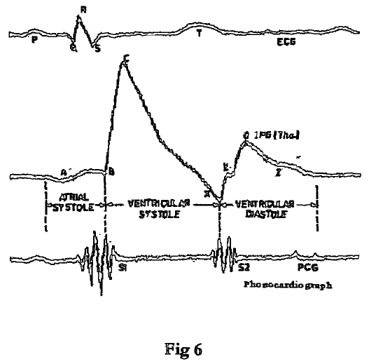

FIG. 6 shows the rate of change of impedance waveform. In order that the cardiac output measurement happens, certain important points need to be selected on the graph. The system provides two modes of measurement, namely, the continuous mode of measurement and the intermittent mode of measurement. Under the continuous mode of measurement, the system automatically selects the important points of ‘A’, ‘B’, ‘C’ and ‘X’ and subsequently calculates the cardiac output measurement. In the intermittent mode of measurement, the clinician operating the system must manually select the points using which the system will subsequently do the calculation of cardiac output.

Algorithm for Selection of Automatic B, C, X Point

### The Algorithm is used for Automatic Selection of Adoptive Thrush hold value, ‘C’, ‘A’, ‘B’, ‘X’ points and Adoptive Search Length from Impedance Cardio Vasography (ICVG) waveform.

### The Thrush Hold and ‘C’ Point Detection.

The Adoptive threshold is selected by Algorithm at 90% of the maximum value of the first 300 samples of input samples of ICVG waveform. The Thrush hold is continuously decayed by the factor as below for comparing with next coming samples (starting from 301^{th}).

Thrush hold=(Thrush hold*0.99)+(sample*0.01).

Once the waveform sample value is more than the Adoptive Thrush hold the decaying is stopped till the waveform value comes below the Adoptive Thrush hold. From the point the samples crosses the Thrush hold and till it comes below the Thrush hold, the values are stored in a buffer and the sample having the Highest value is the ‘C’ point.

### Selection of ‘A’ Point

Point ‘A’ is the least point on raising edge of the ‘C’ peak within Adoptive search width. The search width is considered as a half of ‘C’-‘C’ interval. From ‘C’ point the Algorithm searches for the Minimum value within the Adaptive search width.

### Selection of ‘B’ Point

### Point ‘B’ is the a point which is in between ‘A’ and ‘C’ and which is equivalent to the nearest point at the value of 20% difference between ‘C’ and ‘A’ point value from ‘A’.

### ‘X’ Point Selection

### ‘X’ point is traced as a minimum value of input signal after ‘C’ point on falling edge of waveform with in an adaptive search width.

Manual method of calculation of CO is provided to get accurate CO measurement. This has got two advantages a) when the automated method fails to locate the BCX point at proper the user can manual select the “A” point to get the CO value. B) This also gives facility to select ‘A’ point at different places and do research on the waveform. Here user need to select the ‘A’ point, which is lower, most point left of ‘C’ point. This will enable the unit to select the “C” point (which is the peak point of the waveform) and “X” point, which is lower most point on the right side of the waveform. Once “A” point is selected the unit will use the Algorithm mentioned in the point number one to calculate the CO. This method been validated against the gold standard technique available in the market today. (“Tran thoracic electrical bio-impedance for non-invasive measurement of cardiac output: Comparison with Thermo dilution, Echocardiography and radioisotope method”, A collaborative study between National Institute of Metal Health & Neurosciences, Bangalore, India & Narayan Hrudayalay—A premier Institute of cardiology, Bangalore, India)

In the preferred embodiment of the present invention, the patient information and waveforms can be stored with unique name, can be retrieved for reanalysis and stored. This can also be connected to USB printer and print can be taken on normal A4 size paper. It allows user to store more than 1,000 patient data under the unique name.

The system of the invention provides several advantages over prior art systems and methods. Existing prior art systems also use the same working principle i.e. impedance cardiography. However, these prior art systems use a dedicated PC system for the computation and display of the related waveforms and display of digital values. Such systems while user friendly, need significantly higher level of interconnections between the actual acquisition hardware and the PC. The system of the invention is capable of being hooked up to the subject since it has an in built acquisition hardware along with an industrial PC motherboard, thus avoiding all the extra connections. The system of the invention is stand alone and PC based monitor for measurement of cardiac output using a non invasive technique. The application of the cardiac plethysmography technique was limited in respect of a continuous monitoring system in critically ill patients was restricted due to the complicated inter-connections between the acquisition hardware and PC. This simplifies the level of sophistication required for an operator of the system.

The system of the invention avoids the problems of the prior art since all the necessary hardware for signal acquisition and display has been assembled in a single chassis. The user only needs to hook up the patient to the monitor to get the required waveform on the screen along with the digital values. Also the monitor can be used as a dedicated PC system just by connecting a mouse and a keyboard to it (for which connectors have been provided in the side panel). The overall size of the system is 25% of that of a conventional system (standalone PC and the acquisition hardware). The system also provides for continuous and intermittent modes of measurement of cardiac output measurement which makes the job of the clinician much easier. The system also provides for storage of patients data for subsequent retrieval and re-analysis.

We claim:

1. A cardiac output monitoring system using electrical impedance plythesmography, comprising:
(i) a tetrapolar electrode assembly;
(ii) an analog data acquisition unit coupled to the tetrapolar electrode assembly;
(iii) a processor coupled to the analog data acquisition unit and a display unit; and a
(iv) a means for freezing and/or de-freezing an acquisition waveform wherein current frame on the display unit can be selectively retained.

2. The system as claimed in claim 1 further comprises a means for exporting the physiological data to a spreadsheet readable format.

3. The system as claimed in claim 1, further comprises operating under at least—a calibration mode and a patient mode comprising actual measurements wherein switching is digitally controlled and the input for switching is provided by an optical encoder.

4. The system as claimed in claim 3 wherein the patient mode is configured for implementation in at least one of an Impedance Cardio Vasography and a Non Invasive Cardiac Output.

5. The system as claimed in claim 4 wherein non-invasive cardiac output comprises measurement of one or more amount of blood pumped by heart per minute along with Cardiac Index (CI) cardiac index, Stroke Volume (SV) and systemic vascular resistance (SVR) of a patient.

6. The system as claimed in claim 5 comprises a means for measuring Blood Flow Index (BFI) wherein BFI is used to check the condition of the arteries.

7. The system as claimed in claim 6 comprises a means for checking occlusion in vein blood flow and output a graph indicative of the occlusion.

8. The system as claimed in claim 1, comprises intermittent and continuous measurement of cardiac output measured on at least a plurality of predetermined identified points over the change of impedance with respect to time.

9. The system as claimed in claim 8 wherein during intermittent measurement, at least three points are marked manually using the optical encoder and then the said optical encoder is used to select the calculate menu on the display to calculate the cardiac output.

10. The system as claimed in claim 9 wherein during continuous mode, menu selection is configured to occur automatically.

11. The system as claimed in claim 1, comprises a means to attach a mouse and a key board optionally and be used as a dedicated PC system.

12. The system as claimed in claim 1, further comprises a means to display patient systemic vascular resistance.

13. The system as claimed in claim 1, further comprises a means for connecting the system to an external video system, speaker and microphone.

14. The system as claimed in claim 1, further comprises a hard disk coupled to the processor and a means to replace the hard disk by an on board compact flash memory.

15. The system as claimed in claim 1, further comprises a means to store a patients raw data for retrieval and re-analysis wherein all the data related to analysis are stored and can be viewed by the clinician multiple times.

16. The system as claimed in claim 1, further comprises a means to connect the system to a USB printer to take a print out of all of the patients data in a single page of predetermined size.

17. The system as claimed in claim 1, further having configured with the cal pulse and carrier sine wave generation, and the dZ/dt differentiated waveform generation using digital circuitry.

18. A computer implementable method, comprising:
(i) identifying a plurality of points in a chosen time-frame on the rate of change impedance graph;
(ii) selecting a peak point for the selected window of time frame such that the n/2 points on either side are less than or equal to 75% of the peak point;
(iii) configuring an ‘a’ point as the lower most point on the left side of ‘c’;
(iv) configuring an ‘x’ point as the lower most point on the right side of ‘c’;
(v) configuring a ‘b’ point using ‘a’ and ‘c’ points, at approximately 15% equivalent to the amplitude of difference of ‘c’ and ‘a’ point from ‘a’ point; and
(vi) calculating a beat-to-beat stroke volume using dZ/dtₘₐₓ and Lvet wherein the amplitude value difference of ‘c’ and ‘b is the dZ/dtₘₐₓ and the time difference between ‘x’ and ‘b’ is Lvet.
